(19) United States
(12) Patent Application Publication
(10) Pub. No.: US 2024/0366600 A1
(43) Pub. Date: Nov. 7, 2024

(54) PHARMACEUTICAL COMPOSITIONS AND USE THEREOF

(71) Applicant: SHANGHAI ALLIST PHARMACEUTICALS CO., LTD., Shanghai (CN)

(72) Inventors: Huibing Luo, Shanghai (CN); Qing Li, Shanghai (CN)

(21) Appl. No.: 18/690,221

(22) PCT Filed: Apr. 11, 2022
(86) PCT No.: PCT/CN2022/086053

§ 371 Date: Mar. 7, 2024

(30) Foreign Application Priority Data
Sep. 10, 2021 (WO) PCT/CN2021/117692

(51) Int. Cl. A61K 31/506; A61K 45/06; A61P 35/00

(52) U.S. Cl. CPC A61K 31/506; A61K 45/06; A61P 35/00

(57) ABSTRACT

The present disclosure provides a pharmaceutical composition containing a therapeutically effective amount of furmonertinib or a pharmaceutically acceptable salt thereof and optionally a pharmaceutically acceptable carrier, and use of said pharmaceutical composition in manufacture of a medicament for treating and/or preventing a disease mediated by EGFR exon 20 insertion mutation. The pharmaceutical composition of the present disclosure shows an excellent therapeutic effect on disease mediated by EGFR exon 20 insertion mutation (for example, non-small cell lung cancer (NSCLC)) with little side effects and excellent safety.

### TECHNICAL FIELD

The present disclosure relates to a pharmaceutical composition comprising a therapeutically effective amount of furmonertinib or a pharmaceutically acceptable salt thereof and optionally a pharmaceutically acceptable carrier. The present disclosure also relates to use of the pharmaceutical composition in manufacture of a medicament for treating and/or preventing a disease mediated by epidermal growth factor receptor exon 20 insertion (EGFR Exon 20Ins) mutation (hereinafter sometimes referred to as EGFR Exon 20 insertion mutation). The present disclosure also provides a method of treating and/or preventing a disease mediated by EGFR exon 20 insertion mutation, comprising administering to a patient a therapeutically effective amount of furmonertinib or a pharmaceutically acceptable salt thereof.

### BACKGROUND TECHNOLOGY

Worldwide, lung cancer has always been a malignant tumor with the highest morbidity and mortality and serious harm to human health and life, and 1.76 million people died of lung cancer in 2018 all over the world. Non-small cell lung cancer (NSCLC) comprises approximately 80-85% of all lung cancers. Epidermal Growth Factor Receptor (EGFR) mutation is the most widely studied target in NSCLC; EGFR mutation comprises 17% and 50% in western and Asian NSCLC patients, respectively. Among EGFR genetic mutation, the common sensitive mutations are deletion of exon 19 and point mutation of exon 21 (L858R), comprising 85%-90% of all EGFR mutation. EGFR Exon 20 insertion mutation (EGFR Exon 20Ins) is another major class of mutation in the EGFR mutation, comprising approximately 1-10% of all EGFR mutation types and 1-2% of all NSCLC patients. These insertion mutations are heterogeneous and occur at multiple amino acid positions between 762 and 774, resulting in the insertion of 1-7 amino acids, some of which are locally replicated. To date, 122 EGFR exon 20 insertion mutations have been found, the most common subtypes being D770_N771insX mutation (25.5%), V769_D770insX mutation (24.6%), and H773_V774insX mutation (22.6%).

Over the years, a large number of targeted drugs have been developed for EGFR mutation in NSCLC, such as the first generation of reversible tyrosinase inhibitor (TKI) Gefitinib and Erlotinib for EGFR sensitive mutation, the second generation of irreversible covalent binding inhibitor Afatinib, and the third generation of inhibitor Osimertinib for drug resistant mutation EGFR T790M, which have very good clinical effects. However, the first or second generation of EGFR-TKI is essentially ineffective in treating EGFR exon 20 insertion mutation. In addition, unlike typical EGFR-sensitive mutation and T790M drug-resistant mutation, EGFR exon 20 insertion mutation responds poorly to all FDA-approved EGFR-TKIs (including Osimertinib).

EGFR inhibitors against EGFR exon 20 insertion mutation have also been in clinical development stage at present, such as Poziotinib, TAK-788 (Mobocertinib), and the like, and show potential therapeutic effects in clinical trials. Although these drugs show some efficacy, the efficacy is limited, suggesting that more research is needed to improve the therapeutic efficacy for the EGFR exon 20 insertion mutation. Currently, no small molecule targeted drug against EGFR exon 20 insertion mutation is approved globally, and thus there is a great clinical need.

N-{2-{[2-(dimethylamino)ethyl](methyl)amino}-6-(2,2,2-trifluoroethoxy)-5-{[4-(1-methyl-1H-indol-3-yl)pyrimidin-2-yl]amino}pyridin-3-yl}acrylamide (also referred to as “furmonertinib”) represented by the following formula (I) is described in patent CN105315259B, and the mesilate of the compound of the following formula (I) (also referred to as “furmonertinib mesilate”) is described in patent CN107163026B, and furmonertinib mesilate has been commercialized as a third-generation EGFR-TKI inhibitor, and is mainly used for treating a disease mediated by EGFR-sensitive mutation and T790M drug-resistant mutation. The phase I rising study of furmonertinib mesilate demonstrates that when furmonertinib mesilate is orally taken once per day at a dosage level of 20 mg-240 mg, the tolerance and the safety are good, adverse events of subjects are mild or moderate, dose-limiting toxicity does not occur, and dose-related toxic reaction does not occur; and the phase IIb clinical trial has demonstrated that the oral administration of 80 mg daily dose of furmonertinib mesilate shows a relatively good anti-tumor effect on patients with the EGFR T790M positive advanced non-small cell lung cancer, who has progressive disease after receiving prior systematic anti-tumor therapy, and can alleviate or stabilize the disease progression.

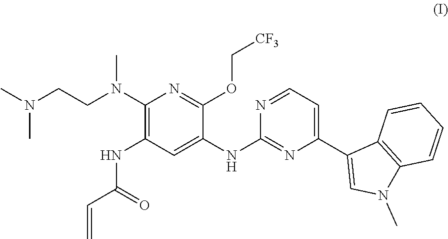 [SMILES: C=CC(=O)Nc1cc(Nc2nccc(-c3cn(C)c4ccccc34)n2)c(OCC(F)(F)F)nc1N(C)CCN(C)C]

SUMMARY

The present disclosure provides, in some embodiments, use of furmonertinib or a pharmaceutically acceptable salt thereof.

In some embodiments, furmonertinib or a pharmaceutically acceptable salt thereof as an active compound can effectively inhibit EGFR exon 20 insertion mutation, and thus, furmonertinib or a pharmaceutically acceptable salt thereof can be used for treating and/or preventing a disease mediated by EGFR exon 20 insertion mutation.

In some embodiments, furmonertinib or a pharmaceutically acceptable salt thereof is useful as an active compound at a certain dose, a disease mediated by EGFR exon 20 insertion mutation, particularly non-small cell lung cancer, can be treated and/or prevented, and the treatment and/or prevention of the disease are/is accompanied by little side effects and is excellent in safety.

More specifically, the present disclosure provides a pharmaceutical composition comprising a therapeutically effective amount of furmonertinib, or a pharmaceutically acceptable salt thereof, and optionally a pharmaceutically acceptable carrier.

The present disclosure also provides use of the above-mentioned pharmaceutical composition of the present disclosure in manufacture of a medicament for treating and/or preventing a disease mediated by the EGFR exon 20 insertion mutation.

The composition of the present disclosure is present in a formulation form of a tablet or a capsule, and in each unit formulation, the content of furmonertinib or a pharmaceutically acceptable salt thereof is 10 mg-400 mg.

When the pharmaceutical composition of the present disclosure is used for treating and/or preventing a disease mediated by EGFR exon 20 insertion mutation, the daily dose of furmonertinib or a pharmaceutically acceptable salt thereof may be 80 mg-400 mg. At this time, by adjusting the amount of the above-mentioned tablets or capsules, the daily dose of furmonertinib or a pharmaceutically acceptable salt thereof can be easily adjusted.

The present disclosure also provides a method of treating and/or preventing a disease mediated by EGFR exon 20 insertion mutation, comprising administering to a patient in need thereof a therapeutically effective amount of furmonertinib or a pharmaceutically acceptable salt thereof.

In the above treatment method of the present disclosure, it is desirable that the daily dose of furmonertinib or a pharmaceutically acceptable salt thereof is 80 mg-400 mg.

The present disclosure also provides a method of treating and/or preventing a disease comprising administering to a patient with positive EGFR exon 20 insertion mutation a therapeutically effective amount of furmonertinib or a pharmaceutically acceptable salt thereof.

The present disclosure also provides a method of treating locally advanced or metastatic non-small cell lung cancer (NSCLC) comprising administering to a patient in need thereof a therapeutically effective amount of furmonertinib or a pharmaceutically acceptable salt thereof.

The present disclosure also provides a method of treating locally advanced or metastatic non-small cell lung cancer (NSCLC) comprising administering to a patient with confirmed positive EGFR exon 20 insertion mutation a therapeutically effective amount of furmonertinib or a pharmaceutically acceptable salt thereof.

The present disclosure also provides a method of treating locally advanced or metastatic non-small cell lung cancer (NSCLC) comprising administering to a patient harboring EGFR exon 20 insertion mutation a therapeutically effective amount of furmonertinib or a pharmaceutically acceptable salt thereof.

The present disclosure also provides a method of treating locally advanced or metastatic non-small cell lung cancer (NSCLC) comprising administering to a patient with confirmed positive EGFR exon 20 insertion mutation who has received no prior systematic anti-tumor therapy a therapeutically effective amount of furmonertinib or a pharmaceutically acceptable salt thereof.

The present disclosure also provides a method of treating locally advanced or metastatic non-small cell lung cancer (NSCLC) comprising administering to a patient with confirmed positive EGFR exon 20 insertion mutation who has progressive disease after receiving prior systematic anti-tumor therapy a therapeutically effective amount of furmonertinib or a pharmaceutically acceptable salt thereof.

In some embodiments, furmonertinib or a pharmaceutically acceptable salt thereof, or a pharmaceutical composition comprising furmonertinib or a pharmaceutically acceptable salt thereof and optionally a pharmaceutically acceptable carrier, exhibits excellent inhibitory activity against EGFR exon 20 insertion mutation, and clinical trial shows that furmonertinib or a pharmaceutically acceptable salt thereof, or a pharmaceutical composition or comprising furmonertinib a pharmaceutically acceptable salt thereof and optionally a pharmaceutically acceptable carrier of the present disclosure, exhibits excellent therapeutic effect against a disease mediated by EGFR exon 20 insertion mutation (for example, non-small cell lung cancer (NSCLC)).

In addition, when furmonertinib or a pharmaceutically acceptable salt thereof, or a pharmaceutical composition comprising furmonertinib or a pharmaceutically acceptable salt thereof and optionally a pharmaceutically acceptable carrier of the present disclosure is used for treating and/or preventing a disease mediated by EGFR exon 20 insertion mutation, the side effect is small and the safety is excellent.

The pharmaceutical composition of the present disclosure can be prepared into a formulation having an appropriate size and an appropriate content of active components by containing furmonertinib or a pharmaceutically acceptable salt thereof in a specific amount.

### DETAILED DESCRIPTION

Embodiments of the present disclosure will be described in more detail below with reference to specific embodiments, but those skilled in the art will appreciate that the specific embodiments described below are merely illustrative of the present disclosure and should not be construed as limiting the scope of the present disclosure. On the contrary, the present disclosure is intended to cover all alternatives, modifications and equivalents, which may be included within the scope of the present disclosure as defined by the appended claims.

Unless otherwise specified, the embodiments of the present disclosure may be combined in any manner, and the conversions, modifications, and changes of the technical solutions obtained thereby are also included in the scope of the present disclosure.

Furmonertinib is a compound known in the prior art, described in particular in patent CN105315259B, with the chemical name: N-{2-{[2-(dimethylamino)ethyl](methyl)amino}-6-(2,2,2-trifluoroethoxy)-5-{[4-(1-methyl-1H-indol-3-yl)pyrimidin-2-yl]amino}pyridin-3-yl}acrylamide; the structural formula is the compound shown in the formula (I).

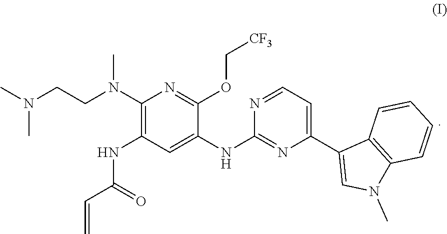 [SMILES: C=CC(=O)Nc1cc(Nc2nccc(-c3cn(C)c4ccccc34)n2)c(OCC(F)(F)F)nc1N(C)CCN(C)C]

In some embodiments, the active component for the treatment of the disease is furmonertinib or a pharmaceutically acceptable salt thereof. Therefore, in some embodiments, furmonertinib or a pharmaceutically acceptable salt thereof may be used alone or may be used by being contained in a composition, in which case the composition may optionally include a pharmaceutically acceptable carrier as desired.

The present disclosure provides a pharmaceutical composition comprising a therapeutically effective amount of furmonertinib or a pharmaceutically acceptable salt thereof and optionally a pharmaceutically acceptable carrier.

“Pharmaceutically acceptable carrier” means one or more compatible solid or liquid fillers or gelatinous materials which are suitable for human use and should be of sufficient purity and sufficiently low toxicity. The carrier is also known as “adjuvant”. “Compatibility” means that each component in the composition can be admixed with the compounds of the present disclosure and with each other without significantly reducing the drug effect of the compounds. Some examples of pharmaceutically acceptable carriers include cellulose and derivatives thereof (such as sodium carboxymethyl cellulose, ethyl cellulose, methyl cellulose, hydroxypropylmethyl cellulose and derivatives thereof, cellulose acetate and derivatives thereof, cellulose acetate, etc.), gelatin, talc, solid lubricants (such as stearic acid, magnesium/calcium stearate, hydrogenated vegetable oil, sodium stearyl fumarate), calcium sulfate, vegetable oils (such as soybean oil, sesame oil, peanut oil, olive oil, etc.), polyols (such as propylene glycol, glycerol, mannitol, sorbitol, etc.), emulsifiers, wetting agents (such as sodium dodecyl sulfate), coloring agents, flavoring agents, stabilizers, antioxidants, preservatives, etc, but not limited thereto.

The pharmaceutical compositions may be prepared by methods well known in the art, such as conventional mixing, dissolving, granulating, dragee-making, levigating, emulsifying, and lyophilizing processes.

The pharmaceutical compositions may be present in the formulation form of a tablet or a capsule, in the formulation, furmonertinib or a pharmaceutically acceptable salt thereof is mixed with at least one pharmaceutically acceptable carrier, in the present disclosure, the carrier is also known as “adjuvant”, said carrier may include but not limited to: (a) fillers or solubilizing agents, for example, microcrystalline cellulose, starch, lactose, sucrose, glucose, mannitol, colloidal silica, calcium hydrogen phosphate, calcium phosphate, calcium sulfate; (b) binders, for example, hydroxypropylmethylcellulose, hydroxypropylcellulose, methylcellulose, alginates, gelatin, polyvinylpyrrolidone, copovidone, sucrose and acacia, corn starch; (c) humectants, for example glycerin and the like; (d) disintegrants, for example, croscarmellose sodium, crospovidone, sodium carboxymethyl starch, colloidal silica, microcrystalline cellulose, potato starch or tapioca starch or corn starch, pregelatinized starch, alginic acid, certain complex silicates and sodium carbonate, ion exchange resins and the like; (e) absorption accelerators, for example, quaternary ammonium compounds, anionic or nonionic surfactants, cyclodextrins, and the like; (f) wetting agents such as cetyl alcohol and glycerol monostearate and the like; (g) adsorbents, for example, kaolin, colloidal silica, ion exchange resins, and the like; and (h) lubricants, for example, talc, calcium stearate, magnesium stearate, solid polyethylene glycols, sodium lauryl sulfate, sodium stearyl fumarate, hydrogenated vegetable oils, and the like, or mixtures thereof. The capsule and the tablet may also contain buffering agent. Tablets and capsules may be coated or microencapsulated with a coating or shell material such as an enteric coating or other materials known in the art.

The term “pharmaceutically acceptable salt” is a salt prepared from furmonertinib and a relatively non-toxic, pharmaceutically acceptable acid or base. Base addition salts may be obtained by contacting furmonertinib with a sufficient amount of a pharmaceutically acceptable base in pure solution or in a suitable inert solvent. Representative base addition salts include, for example, those salts formed with alkali metal, alkaline earth metal, quaternary ammonium cations such as sodium, lithium, potassium, calcium, magnesium, tetramethylquaternary ammonium, tetraethylquaternary ammonium, and the like; amine salts, including salts formed with ammonia (NH₃), primary, secondary or tertiary amines, such as methylamine salts, dimethylamine salts, trimethylamine salts, triethylamine salts, ethylamine salts, and the like. In addition, acid addition salts may be obtained by contacting furmonertinib with a sufficient amount of a pharmaceutically acceptable acid in pure solution or in a suitable inert solvent. The pharmaceutically acceptable acid salt comprises inorganic acid salts such as hydrochloride, sulfate, phosphate, and nitrate; and organic acid salts such as formate, acetate, propionate, methanesulfonate, benzylsulfonate, succinate, citrate, and tartrate. Reference can be specifically made to Berge et al., “Pharmaceutical Salts”, Journal of Pharmaceutical Science 66:1-19 (1977), or “Handbook of Pharmaceutical Salts: Properties, Selection, and Use” (P. Heinrich Stahl and Camille G. Wermuth, ed., Wiley-VCH, 2002).

As used herein, “therapeutically effective amount” refers to a sufficient amount of drug or pharmacologically active agent that is non-toxic but yet achieves the desired effect. The effective amount will vary from person to person, depending on the age, weight and condition of the patient and also on the particular active substance, and an appropriate effective amount in individual cases may be determined by a person skilled in the art in the light of routine test.

As used herein, “active component”, “active substance”, or “active agent” refers to a chemical entity that is effective in treating the disorder, disease, or condition of interest.

As used herein, “patient”, “individual”, or “subject” includes humans, animals, vertebrates, mammals, rodents (e.g., guinea pigs, hamsters, rats, mice), murines (e.g., mice), canines (e.g., dogs), primates, anthropoids (e.g., monkeys or apes), monkeys (e.g., marmosets, baboons), apes (e.g., gorillas, chimpanzees, orangutans, gibbons). In some embodiments, “patient” is a human.

As used herein, “treatment” refers to therapeutic treatment or palliative measures. When specific conditions are involved, treatment refers to: (1) relieving one or more biological manifestations of a disease or a disorder, (2) interfering with (a) one or more points in a biological cascade that causes or contributes to a disorder or (b) one or more biological manifestations of a disorder, (3) ameliorating one or more symptoms, effects, or side effects associated with a disorder, or one or more symptoms, effects, or side effects associated with a disorder or treatment thereof, or (4) slowing the progression of one or more biological manifestations of a disease or a disorder. “Treatment” may also refer to an increase in survival compared to expected survival without receiving the treatment.

As used herein, “prevention” refers to a reduction in the risk of acquiring or developing a disease or a disorder.

In some embodiments, the pharmaceutically acceptable salt of furmonertinib is a mesilate salt of furmonertinib, i.e., furmonertinib mesilate.

In some embodiments, the pharmaceutical composition of the present disclosure is present in the formulation form of a tablet or a capsule.

In some embodiments, in each unit formulation (such as a tablet or a capsule) of the pharmaceutical composition, the content of furmonertinib or a pharmaceutically acceptable salt thereof is 10 mg-400 mg, such as 20 mg-320 mg. As the specific content, for example, it can be 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. In one embodiment, it can be 20 mg, 40 mg, 80 mg, 160 mg, 240 mg or 320 mg, such as 40 mg or 80 mg, such as 40 mg.

In some embodiments, in the pharmaceutical composition, the content of furmonertinib or a pharmaceutically acceptable salt thereof is 80 mg-400 mg, for example, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As exemplary embodiments, it can be 80 mg, 160 mg, 240 mg or 320 mg, such as 80 mg, 160 mg or 240 mg, such as 240 mg.

In some embodiments, when the pharmaceutical composition is used for treating and/or preventing a disease mediated by EGFR exon 20 insertion mutation, the composition is administered to a patient such that the dose of furmonertinib or a pharmaceutically acceptable salt thereof is 80 mg-400 mg. As the specific dose, for example, it can be 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As exemplary embodiments, it can be 80 mg, 160 mg, 240 mg or 320 mg, such as 80 mg, 160 mg or 240 mg, such as 240 mg. In an embodiment of the present disclosure, the dose is a daily dose.

In some embodiments, the content of furmonertinib or a pharmaceutically acceptable salt thereof in the pharmaceutical composition refers to the total amount of furmonertinib or a pharmaceutically acceptable salt thereof in the pharmaceutical composition taken by a patient when said pharmaceutical composition is administered to the patient. For example, when a pharmaceutical composition is present in the formulation form of a tablet or a capsule, the content of furmonertinib or a pharmaceutically acceptable salt thereof in said pharmaceutical composition refers to the total amount of furmonertinib or a pharmaceutically acceptable salt thereof in all formulations (such as tablets or capsules) when the formulations (such as tablets or capsules) are administered.

It will be appreciated by those skilled in the art that when a patient is administered, the daily dose of furmonertinib or a pharmaceutically acceptable salt thereof is not less than the content of furmonertinib or a pharmaceutically acceptable salt thereof in per unit formulation. Those skilled in the art can calculate the total amount of the formulations that is necessary to be administered per day based on the daily dose of furmonertinib or a pharmaceutically acceptable salt thereof and the content of furmonertinib or a pharmaceutically acceptable salt thereof in each unit formulation. For example, when furmonertinib or a pharmaceutically acceptable salt thereof is contained in tablets and the content of furmonertinib or a pharmaceutically acceptable salt thereof in each unit formulation (each tablet) is 40 mg, and when the daily dose of furmonertinib or a pharmaceutically acceptable salt thereof is 240 mg, the total amount of the formulations (tablets) that is necessary to be administered per day is 6 tablets.

In some embodiments, the pharmaceutical composition is administered 1, 2 or 3 times per day, such as once per day, for the treatment and/or prevention of a disease mediated by EGFR exon 20 insertion mutation.

In some embodiments, the pharmaceutical composition may further comprise at least one second therapeutic agent. As the second therapeutic agent, it may be selected from chemotherapeutic drug, targeted antitumor drug, antibody drug and immunotherapeutic drug.

In some embodiments, as said chemotherapeutic drug, the following can be exemplified: one or more of platinum drug (for example oxaliplatin, cisplatin, carboplatin, nedaplatin, dicycloplatin, lobaplatin, triplatinum tetranitrate, phenanthreneplatin, picoplatin, miriplatin, satraplatin), fluoropyrimidine derivative (for example gemcitabine, capecitabine, ancitabine, fluorouracil, tegadifur, doxifluridine, tegafur, carmofur, trifluridine, tegafur), camptothecins (for example camptothecin, hydroxycamptothecine, 9-amino camptothecin, 7-ethyl camptothecin, irinotecan, topotecan), taxels (for example paclitaxel, albumin-bound paclitaxel and docetaxel), vinblastines (vinorelbine, vinblastine, vincristine, vindesine, vinflunine), anthracenes (epirubicin, amycin, rubidomycin, pirarubicin, amrubicin, idarubicin, mitoxantrone, aclarubicin, valrubicin, zorubicin, pixantrone), antibiotics, podophyllums, antimetabolite antitumor drug, pemetrexed, carmustine, melphalan, etoposide, teniposide, mitomycin, iphosphamide, cyclophosphamide, azacitidine, methotrexate, bendamustine, liposome amycin, actinomycin D (dactinomycin), bleomycin, pingyangmycin, temozolomide, decarbazine, peplomycin, eribulin, plinabulin, sapacitabine, treosulfan, 153Sm-EDTMP, and encequidar.

In some embodiments, said second therapeutic agent is one or more of platinum drug, and said platinum drug includes, but is not limited to Cisplatin, Carboplatin, Nedaplatin, oxaliplatin, triplatinum tetranitrate, phenanthreneplatin, picoplatin, satraplatin, miriplatin, Lobaplatin and the like.

In some embodiments, said chemotherapeutic drug is selected from one or more of etoposide, irinotecan, cisplatin, carboplatin, lobaplatin, nedaplatin, topotecan, paclitaxel, docetaxel, temozolomide, vinorelbine, gemcitabine, cyclophosphamide, amycin, vincristine, bendamustine, pharmorubicin, methotrexate, amrubjcin, tegafur, gimeracil, oteracil, tegafur.

In some embodiments, as the targeted antitumor drug, protein kinase inhibitors can be enumerated. Among them, the protein kinase inhibitors include but are not limited to tyrosine kinase inhibitors, serine and/or threonine kinase inhibitors, and poly ADP-ribose polymerase (PARP) inhibitors. The targets of the inhibitors include but are not limited to Fascin-1 protein, HDAC (histone deacetylase), Proteasome, CD38, SLAMF7 (CS1/CD319/CRACC), RANKL, EGFR (epidermal growth factor receptor), anaplastic lymphoma (ALK), METgene, ROS1gene, HER2gene, RETgene, BRAFgene, PI3K signal pathway, DDR2 (discoidin domain receptor 2) gene, FGFR1 (fibroblast growth factor receptor 1), NTRK1 (neurotrophic tyrosine kinase type 1 receptor) gene, and KRASgene. The targets of the targeted antitumor drug also include COX-2 (epoxidase-2), APE1 (apurinic-apyrimidinic endonuclease), VEGFR (vascular endothelial growth factor receptor), CXCR-4 (chemokine receptor-4), MMP (matrix metalloproteinase), IGF-1R (insulin-like growth factor receptor), Ezrin, PEDF (pigmented epithelial derived factor), AS, ES, OPG (bone protective factor), Src, IFN, ALCAM (activated leukocyte cell adhesion molecule), HSP, JIP1, GSK-3β (Glycogen Synthetic Kinase 3β), CyclinD1 (cell cycle regulator protein), CDK4 (cyclin-dependent kinase), TIMP1 (tissue metalloproteinase inhibitor), THBS3, PTHR1 (parathyroid hormone-related protein receptor 1), TEM7 (human tumor vascular endothelial marker 7), COPS3, and cathepsin K. The targeted antitumor drug that can be enumerated includes but is not limited to one or more of Imatinib, Sunitinib, Nilotinib, bosutinib, Saracatinib, Pazopanib, Trabectedin, Regorafenib, Cediranib, Bortezomib, Panobinostat, Carfilzomib, Ixazomib, apatinib, Erlotinib, Afatinib, Crizotinib, Ceritinib, Vemurafenib, Dabrafenib, Cabozantinib, Gefitinib, Dacomitinib, Almonertinib, Osimertinib, Olmutinib, Alectinib, Brigatinib, Lorlatinib, Trametinib, Larotrectinib, icotinib, Lapatinib, Vandetanib, Selumetinib, Sorafenib, Olmutinib, Savolitinib, Fruquintinib, Entrectinib, Dasatinib, Ensartinib, Lenvatinib, itacitinib, Pyrotinib, Binimetinib, Erdafitinib, Axitinib, Neratinib, Cobimetinib, Acalabrutinib, Famitinib, Masitinib, Ibrutinib, Anlotinib, rociletinib, nintedanib, Revlimid, LOXO-292, Vorolanib, bemcentinib, capmatinib, entrectinib, TAK-931, ALT-803, palbociclib, famitinib L-malate, LTT-462, BLU-667, ningetinib, tipifarnib, poziotinib, DS-1205c, capivasertib, SH-1028, Metformin, seliciclib, OSE-2101, APL-101, berzosertib, idelalisib, lerociclib, ceralasertib, PLB-1003, tomivosertib, SKLB-1028, D-0316, LY-3023414, allitinib, MRTX-849, AP-32788, AZD-4205, lifirafenib, vactosertib, mivebresib, napabucasin, sitravatinib, TAS-114, molibresib, CC-223, rivoceranib, CK-101, LXH-254, simotinib, GSK-3368715, TAS-0728, masitinib, tepotinib, HS-10296, AZD-4547, merestinib, olaptesed pegol, galunisertib, ASN-003, gedatolisib, defactinib, lazertinib, CKI-27, S-49076, BPI-9016M, RF-A-089, RMC-4630, AZD-3759, antroquinonol, SAF-189s, AT-101, TTI-101, naputinib, LNP-3794, HH-SCC-244, ASK-120067, CT-707, epitinib succinate, tesevatinib, SPH-1188-11, BPI-15000, copanlisib, niraparib, olaparib, veliparib, talazoparib tosylate, DV-281, Siremadlin, Telaglenastat, MP-0250, GLG-801, ABTL-0812, bortezomib, tucidinostat, vorinostat, resminostat, epacadostat, tazemetostat, entinostat, mocetinostat, quisinostat, LCL-161, and KML-001. In some embodiments, the targeted antitumor drug is one or more of Sorafenib, Erlotinib, Afatinib, Crizotinib, Ceritinib, Vemurafenib, Dabrafenib, Cabozantinib, Gefitinib, Dacomtinib, Osimertinib, Alectinib, Brigatinib, Lorlatinib, Trametinib, Larotrectinib, Icotinib, Lapatinib, Vandetanib, Selumetinib, Olmutinib, Savolitinib, Fruquintinib, Entrectinib, Dasatinib, Ensartinib, Lenvatinib, Itacitinib, Pyrotinib, Binimetinib, Erdafitinib, Axitinib, Niratinib, Cobimetinib, Acalabrutinib, Famitinib, Masitinib, Ibrutinib, Anlotinib, Nintedanib.

In some embodiments, the second therapeutic agent is an antibody drug. Among others, the targets aimed by the antibody drug include but are not limited to any one or more of PD-1, PD-L1, cytotoxic T-lymphocyte antigen 4 (CTLA-4), platelet-derived growth factor receptor α (PDGFR-α), vascular endothelial growth factor (VEGF), human epidermal growth factor receptor-2 (HER2), epidermal growth factor receptor (EGFR), ganglioside GD2, B-cell surface protein CD20, B-cell surface protein CD52, B-cell surface protein CD38, B-cell surface protein CD319, B-cell surface protein CD30, and B-cell surface protein CD19/CD3.

In some embodiments, the antibody drug is an inhibitor for the interaction between the PD-1 receptor and its ligand PD-L1; in an embodiment of the present disclosure, the antibody drug is cytotoxic T-lymphocyte antigen 4 inhibitor. In an embodiment of the present disclosure, the antibody drug is platelet-derived growth factor receptor α (PDGFR-α) inhibitor.

In some embodiments, the inhibitor for the interaction between the PD-1 receptor and its ligand PD-L1 is an antibody or its antigen-binding portion that binds to the programmed death receptor 1 (PD-1) and/or inhibits the activity of PD-1, or an antibody or its antigen-binding portion that binds to the programmed death ligand 1 (PD-L1) and/or inhibits the activity of PD-L1, for example, an anti-PD-1 antibody or an anti-PD-L1 antibody. In an embodiment of the present disclosure, the antibody or its antigen-binding portion is (a) an anti-PD-1 monoclonal antibody or its antigen-binding fragment, which specifically binds to human PD-1 and blocks the binding between human PD-L1 and human PD-1; or (b) an anti-PD-L1 monoclonal antibody or its antigen-binding fragment, which specifically binds to human PD-L1 and blocks the binding between human PD-L1 and human PD-1.

In some embodiments, the anti-PD-1 or PD-L1 antibody is an anti-PD-1 or PD-L1 monoclonal antibody.

In some embodiments, the anti-PD-1 or PD-L1 antibody is a human antibody or a murine antibody.

In some embodiments, the anti-PD-1 antibody can be selected from any one or more of Nivolumab, Pembrolizumab, Durvalumab, Toripalimab (JS-001), Sintilimab (IBI308), Camrelizumab, Tislelizumab (BGB-A317), Geptanolimab (GB226), Lizumab (LZM009), HLX-10, BAT-1306, AK103 (HX008), AK104 (Akesobio), CS1003, SCT-110A, F520, SG001, and GLS-010.

In some embodiments, the anti-PD-L1 antibody can be selected from any one or more of Atezolizumab, Avelumab, Durvalumab, KL-A167, SHR-1316, BGB-333, JS003, STI-A1014 (ZKAB0011), KN035, MSB2311, HLX-20, and CS-1001.

In some embodiments, the anti-PD-1 antibody is Toripalimab.

In an embodiment of the present disclosure, the anti-PD-1 antibody is Pembrolizumab.

In some embodiments, the cytotoxic T-lymphocyte antigen 4 (CTLA-4) inhibitor is an anti-CTLA-4 antibody, in an embodiment of the present disclosure, the anti-CTLA-4 antibody is an anti-CTLA-4 monoclonal antibody.

In some embodiments, the anti-CTLA-4 antibody can be selected from any one or more of Ipilimumab, Tremelimumab, AGEN-1884, BMS-986249, BMS-986218, AK-104, and IBI310.

In some embodiments, the anti-CTLA-4 antibody is Ipilimumab.

In some embodiments, the platelet-derived growth factor receptor α (PDGFR-α) inhibitor is an anti-PDGFR α antibody. In an embodiment of the present disclosure, the anti-PDGFRα antibody is an anti-PDGFRα monoclonal antibody.

In some embodiments, the anti-PDGFRα antibody is Olaratumab.

In some embodiments, the antibody drug can also include, but are not limited to any one or more of Bevacizumab, Ramucirumab, Pertuzumab, Trastuzmab, Cotuximab, Nimotuzumab, Panitumumab, Necitumumab, Dinutuximab, Rituximab, Ibritumomab, Ofatumumab, Obinutuzumab, Alemtuzumab, Daratumumab, Gemtuzumab, Elotuzumab, Brentuximab, Inotuzumab Ozogamicin, Blinatumomab.

In some embodiments, as immunotherapeutic drug, the following can be enumerated: one or more interferon (interferon α, interferon α-1b, interferon α-2b), interleukin, temsirolimus, everolimus, ridaforolimus, and temsirolimus.

In some embodiments, when a second therapeutic agent is used, the amount of the second therapeutic agent can be adjusted as desired by those skilled in the art.

In some embodiments, use of the above-mentioned pharmaceutical composition in manufacture of a medicament for treating and/or preventing a disease mediated by the EGFR exon 20 insertion mutation is provided.

In some embodiments, in the use described herein, the pharmaceutically acceptable salt of furmonertinib is a mesilate salt of furmonertinib, i.e., furmonertinib mesilate.

In some embodiments, in the use described herein, the pharmaceutical composition of the present disclosure is present in the formulation form of a tablet or a capsule.

In some embodiments, in the use described herein, in each unit formulation (such as a tablet or a capsule), the content of furmonertinib or a pharmaceutically acceptable salt thereof is 10 mg-400 mg, such as 20 mg-320 mg. As the specific content, it can be for example 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the exemplary specific content, it can be 20 mg, 40 mg, 80 mg, 160 mg, 240 mg or 320 mg, such as 40 mg or 80 mg, such as 40 mg.

In some embodiments, in the use described herein, in said pharmaceutical composition, the content of furmonertinib or a pharmaceutically acceptable salt thereof is 80 mg-400 mg, for example 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the exemplary content, it can be 80 mg, 160 mg, 240 mg or 320 mg, such as 80 mg, 160 mg or 240 mg, such as 240 mg.

In some embodiments, in the use described herein, the pharmaceutical composition is administered to a patient such that the dose of furmonertinib or a pharmaceutically acceptable salt thereof is 80 mg-400 mg. As the specific dose, it can be for example 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the exemplary dose, it can be 80 mg, 160 mg, 240 mg or 320 mg, such as 80 mg, 160 mg or 240 mg, such as 240 mg. In an embodiment of the present disclosure, the dose is a daily dose.

In some embodiments, in the use described herein, the content of furmonertinib or a pharmaceutically acceptable salt thereof in the pharmaceutical composition refers to the total amount of furmonertinib or a pharmaceutically acceptable salt thereof in the pharmaceutical composition taken by a patient when said pharmaceutical composition is administered to the patient. For example, when a pharmaceutical composition is present in the formulation form of a tablet or a capsule, the content of furmonertinib or a pharmaceutically acceptable salt thereof in said pharmaceutical composition refers to the total amount of furmonertinib or a pharmaceutically acceptable salt thereof in all formulations (such as tablets or capsules) when the formulations (such as tablets or capsules) are administered.

It will be appreciated by those skilled in the art that in the use described herein, when a patient is administered, the daily dose of furmonertinib or a pharmaceutically acceptable salt thereof is not less than the content of furmonertinib or a pharmaceutically acceptable salt thereof in per unit formulation. Those skilled in the art can calculate the total amount of the formulations that is necessary to be administered per day based on the daily dose of furmonertinib or a pharmaceutically acceptable salt thereof and the content of furmonertinib or a pharmaceutically acceptable salt thereof in each unit formulation. For example, when furmonertinib or a pharmaceutically acceptable salt thereof is contained in tablets and the content of furmonertinib or a pharmaceutically acceptable salt thereof in each unit formulation (each tablet) is 40 mg, and when the daily dose of furmonertinib or a pharmaceutically acceptable salt thereof is 240 mg, the total amount of the formulations (tablets) that is necessary to be administered per day is 6 tablets.

In some embodiments, the disease mediated by EGFR exon 20 insertion mutation is cancer, for example lung cancer, and further can be non-small cell lung cancer (NSCLC).

In some embodiments, the disease mediated by EGFR exon 20 insertion mutation is locally advanced non-small cell lung cancer or metastatic non-small cell lung cancer.

In some embodiments, the disease mediated by EGFR exon 20 insertion mutation is a treatment-naive non-small cell lung cancer or a previously-treated non-small cell lung cancer.

As used herein, the term “treatment-naive” refers to a condition where before receiving the treatment with furmonertinib or a pharmaceutically acceptable salt thereof of the present disclosure, the treatment with another therapeutic agent (including but not limited to chemotherapeutic drug, targeted antitumor drug, antibody drug or immunotherapeutic drug) has not been used, or a condition where no systematic anti-tumor therapy has been taken. As used herein, the term “previously-treated” refers to a condition where before receiving the treatment with furmonertinib or a pharmaceutically acceptable salt thereof of the present disclosure, the treatment with another therapeutic agent (including but not limited to chemotherapeutic drug, targeted antitumor drug, antibody drug or immunotherapeutic drug) has been used, or a condition where a systematic anti-tumor therapy has been taken, but afterwards the disease has progressed. In the case of “previously-treated”, the patient may have developed the resistance to other therapeutic agents, or may not develop the drug resistance.

In some embodiments, the EGFR exon 20 insertion mutation is characterized by a plurality of amino acid insertion mutations in the area of amino acids 762-774 of the EGFR protein, that is to say, the exon 20 insertion mutation site is located in the area of amino acids 762-774, such as the EGFR exon 20 insertion mutation is at least one selected from EGFR D770_N771insX mutation, EGFR V769_D770insX mutation, EGFR H773_V774insX mutation and EGFR P772_H773insX mutation, such as, the EGFR exon 20 insertion mutation is at least one selected from EGFR D770_N771insSVD, EGFR V769_D770insASV, EGFR H773_V774insNPH and EGFR D770_N771insNPG.

In some embodiments, in the use described herein, the pharmaceutical composition may further comprise at least one second therapeutic agent. In the use described herein, the second therapeutic agent can be selected from chemotherapeutic drug, targeted antitumor drug, antibody drug and immunotherapeutic drug.

In some embodiments, in the use described herein, the second therapeutic agent is the above-mentioned the second therapeutic agent of the present disclosure.

In some embodiments, a method of treating and/or preventing a disease mediated by EGFR exon 20 insertion mutation is provided, comprising administering to a patient a therapeutically effective amount of furmonertinib or a pharmaceutically acceptable salt thereof.

In some embodiments, a method of treating and/or preventing a disease comprising administering to a patient with positive EGFR exon 20 insertion mutation a therapeutically effective amount of furmonertinib or a pharmaceutically acceptable salt thereof is provided.

In some embodiments, a method of treating locally advanced or metastatic non-small cell lung cancer (NSCLC) is provided, comprising administering to a patient in need thereof a therapeutically effective amount of furmonertinib or a pharmaceutically acceptable salt thereof.

In some embodiments, a method of treating locally advanced or metastatic non-small cell lung cancer (NSCLC) is provided, comprising administering to a patient with confirmed positive EGFR exon 20 insertion mutation a therapeutically effective amount of furmonertinib or a pharmaceutically acceptable salt thereof.

In some embodiments, a method of treating locally advanced or metastatic non-small cell lung cancer (NSCLC) is provided, comprising administering to a patient harboring EGFR exon 20 insertion mutation a therapeutically effective amount of furmonertinib or a pharmaceutically acceptable salt thereof.

In some embodiments, a method of treating locally advanced or metastatic non-small cell lung cancer (NSCLC) is provided, comprising administering to a patient with confirmed positive EGFR exon 20 insertion mutation who has received no prior systematic anti-tumor therapy a therapeutically effective amount of furmonertinib or a pharmaceutically acceptable salt thereof.

In some embodiments, a method of treating locally advanced or metastatic non-small cell lung cancer (NSCLC) is provided, comprising administering to a patient with confirmed positive EGFR exon 20 insertion mutation who has progressive disease after receiving prior systematic anti-tumor therapy a therapeutically effective amount of furmonertinib or a pharmaceutically acceptable salt thereof.

In some embodiments of the treatment method, furmonertinib or a pharmaceutically acceptable salt thereof is administrated at a dose of 80 mg-400 mg. As the specific dose, it can be for example 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the exemplary dose, it can be 80 mg, 160 mg, 240 mg or 320 mg, such as 80 mg, 160 mg or 240 mg, such as 240 mg. In an embodiment of the present disclosure, the dose is a daily dose.

In some embodiments of the treatment method, the frequency at which furmonertinib or a pharmaceutically acceptable salt thereof is administered to a patient is 1 time per day (qd), 2 times per day (bid), or 3 times per day (tid), such as 1 time per day.

In some embodiments of the treatment method, furmonertinib or a pharmaceutically acceptable salt thereof is administered to a patient under fasted state, such as under fasted state in the morning.

In some embodiments of the treatment method, furmonertinib or a pharmaceutically acceptable salt thereof is orally administered to a patient.

In some embodiments, in the method described herein, furmonertinib is administered in the form of a mesilate salt.

In some embodiments of the treatment method, furmonertinib or a pharmaceutically acceptable salt thereof is administered in the formulation form of a tablet or a capsule.

In some embodiments of the treatment method, furmonertinib or a pharmaceutically acceptable salt thereof is administered to a patient in the form of each unit formulation. By adjusting the amount of unit formulation, the daily dose of furmonertinib or a pharmaceutically acceptable salt thereof is in the above range.

In some embodiments of the treatment method, in each unit formulation (such as a tablet or a capsule), the content of said furmonertinib or a pharmaceutically acceptable salt thereof is 10 mg-400 mg, such as 20 mg-320 mg. As the specific content, it can be for example mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 10 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the exemplary specific content, it can be 20 mg, 40 mg, 80 mg, 160 mg, 240 mg or 320 mg, such as 40 mg or 80 mg, such as 40 mg.

It will be appreciated by those skilled in the art that when a patient is administered, the daily dose of furmonertinib or a pharmaceutically acceptable salt thereof is not less than the content of furmonertinib or a pharmaceutically acceptable salt thereof in per unit formulation. Those skilled in the art can calculate the total amount of the formulations that is necessary to be administered per day based on the daily dose of furmonertinib or a pharmaceutically acceptable salt thereof and the content of furmonertinib or a pharmaceutically acceptable salt thereof in each unit formulation. For example, when furmonertinib or a pharmaceutically acceptable salt thereof is contained in tablets and the content of furmonertinib or a pharmaceutically acceptable salt thereof in each unit formulation (each tablet) is 40 mg, and when the daily dose of furmonertinib or a pharmaceutically acceptable salt thereof is 240 mg, the total amount of the formulations (tablets) that is necessary to be administered per day is 6 tablets.

In some embodiments of the treatment method, at least one second therapeutic agent can be further administered to a patient. In some embodiments of the treatment method, as the second therapeutic agent, it can be selected from chemotherapeutic drug, targeted antitumor drug, antibody drug and immunotherapeutic drug.

In some embodiments of the treatment method, the second therapeutic agent is the above-mentioned the second therapeutic agent of the present disclosure.

In some embodiments of the treatment method, the disease is cancer, for example lung cancer, and further can be non-small cell lung cancer (NSCLC).

In some embodiments of the treatment method, furmonertinib or a pharmaceutically acceptable salt thereof is administered to a patient before or after surgical resection of tumor.

In some embodiments of the treatment method, the disease is locally advanced non-small cell lung cancer or metastatic non-small cell lung cancer.

In some embodiments of the treatment method, the disease is a treatment-naive non-small cell lung cancer or a previously-treated non-small cell lung cancer.

In some embodiments of the treatment method, the EGFR exon 20 insertion mutation is characterized by a plurality of amino acid insertion mutations in the area of amino acids 762-774 of the EGFR protein, that is to say, the exon 20 insertion mutation site is located in the area of amino acids 762-774, such as the EGFR exon 20 insertion mutation is at least one selected from EGFR D770_N771insX mutation, EGFR V769_D770insX mutation, EGFR H773_V774insX mutation and EGFR P772_H773insX mutation, such as, the EGFR exon 20 insertion mutation is at least one selected from EGFR D770_N771insSVD, EGFR V769_D770insASV, EGFR H773_V774insNPH and EGFR D770_N771insNPG.

In some embodiments of the treatment method, the patient is a human patient.

In some embodiments of the treatment method, the patient is between age 18 and 75.

In some embodiments of the treatment method, the patient has histologically or cytopathologically confirmed primary non-small cell lung cancer (NSCLC) with predominant non-squamous cell histology prior to the start of treatment with furmonertinib or a pharmaceutically acceptable salt thereof.

In some embodiments of the treatment method, the patient has radiological disease progression following the last anti-tumor therapy prior to the start of treatment with furmonertinib or a pharmaceutically acceptable salt thereof.

In some embodiments of the treatment method, the patient has documented positive EGFR exon 20 insertion mutation by laboratory test prior to the start of treatment with furmonertinib or a pharmaceutically acceptable salt thereof.

In some embodiments of the treatment method, the patient has locally advanced non-small cell lung cancer or metastatic non-small cell lung cancer and is confirmed to have radiological or pathological disease progression during or after the last systematic anti-tumor therapy prior to the start of treatment with furmonertinib or a pharmaceutically acceptable salt thereof.

In some embodiments the treatment method, the patient has locally advanced non-small cell lung cancer or metastatic non-small cell lung cancer and has received no prior systematic anti-tumor therapy prior to the start of treatment with furmonertinib or a pharmaceutically acceptable salt thereof.

In some embodiments of the treatment method, the patient has at least one measurable lesion prior to the start of treatment with furmonertinib or a pharmaceutically acceptable salt thereof.

In some embodiments of the treatment method, the patient has adequate organ function as shown by laboratory test prior to the start of treatment with furmonertinib or a pharmaceutically acceptable salt thereof.

In some embodiments of the treatment method, the patient is subjected to an ECOG PS (Eastern Cooperative Oncology Group performance status) score test, such as an ECOG PS score of 0-1, prior to the start of treatment with furmonertinib or a pharmaceutically acceptable salt thereof.

In some embodiments, the treatment method has an acceptable safety profile.

In some embodiments, the treatment method can provide the therapeutic efficacy of partial response (PR).

In some embodiments, the treatment method can provide the therapeutic efficacy of stable disease (SD).

In some embodiments, the treatment method can provide tumor shrinkage in target lesions.

In some embodiments, the treatment method, tumor shrinkage in target lesions is provided, as evaluated by tumor radiological examination, such as computed tomography (CT) and/or magnetic resonance imaging (MRI).

### BRIEF DESCRIPTION OF THE DRAWINGS

FIG. 1. The curves of tumor volume change in Test Example 3.

### EXAMPLES

### Explanation of Abbreviations

PR: Partial response; SD: Stable disease; Naive: Treatment-naive; DCR: Disease control rate; DOR: Duration of response; DepOR: Depth of response; PFS: Progression-free survival; OS: Overall survival; CNS ORR: Central nervous system objective response rate; CTCAE: Common terminology criteria for adverse events; RECIST1.1: Response Evaluation Criteria in Solid Tumors guidelines (version 1.1); ctDNA: Circulating tumor DNA; NYHA: New York Heart Association; AJCC: American Joint Committee on Cancer; CYP3A4: Cytochrome P450 3A4; QTc: Corrected QT Interval.

### I. Preparation Examples

Preparation of furmonertinib mesilate, 40 mg, standard tablet

Formula: furmonertinib mesilate 46.76 mg, microcrystalline cellulose 44.73 mg, lactose 68.2 mg, croscarmellose sodium 13 mg, polyethylene glycol 4000 17.8 mg, colloidal silica 10.9 mg, sodium stearyl fumarate 2.7 mg, sodium chloride 8.67 mg, and 40 mg furmonertinib contained therein.

Process: sieving adjuvants and the active pharmaceutical ingredient for pretreatment and mixing uniformly, adding an appropriate amount of Polyethylene Glycol 4000 for wet granulation, sieving out for wet granulation, drying the wet granules, sieving out for granulation, adding colloidal silica and sodium stearyl fumarate and mixing uniformly, and then tabletting to obtain tablets.

### II. Activity Examples

### 1. Cell Viability

### Test Example 1: Proliferation Inhibitory Activity on the Adherent Cell of Human Skin Cancer A431 (Wild Type EGFR)

The proliferation inhibitory activity of a compound (furmonertinib mesilate) against the adherent cell of human skin cancer A431 expressing wild-type EGFR protein in vitro was determined by the sulforhodamine B method (SRB method).

Cell source: A431 cells were purchased from Shanghai Dobio Biology Technology Inc.

A431 cells were cultivated in DMEM complete culture medium containing 10% fetal bovine serum. A431 cells in the logarithmic growth phase were taken and inoculated in 96-well plates according to the cell density of 5000 cells/135 μL of complete culture medium/well, and the plates were placed in a constant temperature incubator containing 5% CO₂ at 37° C. and cultivated for 24 hours to ensure the complete adherence of the cells. The compound was dissolved in dimethyl sulfoxide (DMSO) in advance to prepare a 10 mM stock solution, and then the compound was successively diluted with DMSO and the complete culture medium. The 96-well plates inoculated with the cells were taken out, and one of the plates was taken alone as a growth-free control group (a culture medium control group with no cell growth at 0 hour); for the other 96-well plates, 15 μL of different concentrations of the compound were added to each well to achieve final concentrations of 2500, 625, 156.25, 39.06, 9.77, 2.44, 0.61, 0.15, 0.04, 0.01 nM, three duplicate wells were set for each compound concentration, and a negative control (a cell-containing, compound-free culture medium control) was set, and the DMSO concentration in each well was 0.5%.

The cultivation of the set-aside growth-free control group was immediately terminated, and the other 96-well plates were further placed in an incubator containing 5% CO₂ at 37° C. and cultivated for 72 hours and then terminated. The termination of the cultivation method was as follows: 50 μL of pre-cooled (4° C.) 50% aqueous trichloroacetic acid solution was added to each well, held at 4° C. for 1 hour, washed with purified water for at least 5 times, and naturally dried in air or dried at 60° C. in an oven.

A 4 mg/mL SRB solution was formulated in purified water containing 1% glacial acetic acid. 100 μL of the SRB solution was added to each well, and stained at room temperature for 1 hour. The supernatant was discarded, and the residue was washed with 1% glacial acetic acid for at least 5 times to remove the nonspecific binding, and dried for use. 150 μL of 10 mM trihydroxymethylaminomethane hydrochloride solution (Tris-HCl solution) was added to each well for dissolution, and the photometric density value (OD value) at a wavelength of 510nm was measured, and the data was collated to calculate the cell proliferation inhibition rate.

$$Cell proliferation inhibition rate = [ ( {OD}_{72 - hour negative control group} - {OD}_{72 - hour compound administration group} ) / {OD}_{72 - hour negative control group} - {OD}_{growth - free control group} ) ] \times 100 \% .$$

The data were analyzed using GraphPad Prism 8.3 software, fitted with nonlinear S-curve regression to give a dose-effect curve, and IC₅₀ values were calculated therefrom, as shown in Table 1.

TABLE 1

| | A431 |
|---|---|
| Compound | IC₅₀(nM) |
| Furmonertinib mesilate | 109.9 |

### Test Example 2: Proliferation Inhibition Activity on Ba/F3 EGFR D770_N771insSVD, Ba/F3 EGFR V769_D770insASV, Ba/F3 EGFR H773_V774insNPH Stable Transfected Cells

The proliferation inhibition activity of the compound (furmonertinib mesilate) on Ba/F3 EGFR D770_N771insSVD, Ba/F3 EGFR V769_D770insASV, Ba/F3 EGFR H773_V774insNPH cells stably expressing EGFR exon 20 insertion in mouse pro-B cells Ba/F3 in vitro was determined by a CellTiter Glo method.

Cell sources: Ba/F3 EGFR D770_N771insSVD, Ba/F3 EGFR V769_D770insASV, and Ba/F3 EGFR H773_V774insNPH cells, commercially available from Shanghai WuXi AppTec New Drug Development Co. Ltd.

Ba/F3 EGFR D770_N771insSVD, Ba/F3 EGFR V769_D770insASV, and Ba/F3 EGFR H773_V774insNPH cells were cultivated in a RPMI1640 complete culture medium containing 10% fetal bovine serum. Ba/F3 EGFR D770_N771insSVD, Ba/F3 EGFR V769_D770insASV, Ba/F3 EGFR H773_V774insNPH cells in the logarithmic growth phase were taken and inoculated in 384-well plates according to the cell density of 2000 cells/50 μL of complete culture medium/well, and the plates were placed in a constant temperature incubator containing 5% CO₂ at 37° C. and cultivated for 24 hours. The compound was dissolved in dimethyl sulfoxide (DMSO) in advance to prepare a 10 mM stock solution, and then the compound was successively diluted with DMSO and the complete culture medium. The 384-well plates inoculated with the cells were taken out, the compound was added with Tecan HP D300 to achieve final concentrations of 2500, 625, 156.25, 39.06, 9.77, 2.44, 0.61, 0.15, and 0.04 nM, two duplicate wells were set for each compound concentration, and a cell-free culture medium control group was set, and a control group of cell with 0.2% DMSO added was set.

After continuing the cultivation in 5% CO₂ at 37° C. for 72 hours, 25 μL CellTiter-Glo reagent (luciferase ATP bioluminescence detection reagent, commercially available from Promega, Cat #G7573) was added to each well, shaking was carried out at 1000 rpm for 30 seconds, the incubation was carried out at room temperature for 10 minutes, and after the luminescence intensity became stable, the luminescence intensity (Lum) was measured with a microplate reader. The inhibition rate of the compound on cell proliferation at each concentration was calculated.

Cell proliferation inhibition rate=(Lum_{Max}−Lum_{compound})/(Lum_{Max}−Lum_{Min})×100% (Lum_{Max} is the maximum luminescence intensity, Lum_{compound} is the luminescence intensity of the compound, and Lum_{Min} is the minimum luminescence intensity).

The data were analyzed by using XL-fit 5.0 software and fitted with the non-linear S-curve regression to give a dose-effect curve, and IC₅₀ values were calculated therefrom, as shown in Table 2.

TABLE 2

| | Furmonertinib mesylate |
|---|---|
| Stable transfected cells | IC₅₀ (nM) |
| Ba/F3 EGFR D770_N771insSVD | 11 |
| Ba/F3 EGFR V769_D770insASV | 14 |
| Ba/F3 EGFR H773_V774insNPH | 20 |

The result showed that furmonertinib mesilate had good proliferation inhibition activity on Ba/F3 EGFR D770_N771insSVD, Ba/F3 EGFR V769_D770insASV, and Ba/F3 EGFR H773_V774insNPH stable transfected cells.

### 2. In Vivo Study

### Test Example 3: Testing the Anti-Tumor Effect of Furmonertinib Mesilate in HuPrime® Lung Cancer LU0387 Tumor Model

This study was used for evaluating and testing the antitumor effect of furmonertinib mesilate in HuPrime® lung cancer LU0387 (having EGFR H773_V774insNPH mutation) subcutaneous xenografted BALB/c female nude mouse animal models.

Experimental animals: BALB/c nude mice, female, 7-8 weeks (mouse week-old when tumor cells were inoculated), and weighing 18.1-24.7 g, purchased from Jiangsu GemPharmatech Co. Ltd.

Animal modeling and random grouping: tumor tissues were collected from HuPrime® lung cancer xenograft model LU0387 tumor-bearing mice, cut into tumor blocks with diameter of 2-3 mm, and inoculated in Balb/c nude mouse right front scapular subcutaneous, and the inoculation date was 16 Jun. 2020. When the average tumor volume was 197.56 mm³, the animals were randomly grouped according to the tumor size, the grouping date was 15 Jul. 2020, namely the 29th day (Day 29) after the tumor was switched over. The grouping day was defined as Day 0.

The experimental scheme: BALB/c nude mice were subcutaneously inoculated with HuPrime® model LU0387 tumor blocks, and a human lung cancer subcutaneous graft tumor model was established. The experiment was divided into 20 mg/kg group, 30 mg/kg group, and 50 mg/kg group of furmonertinib mesilate and a vehicle group, wherein each group contained 8 animals, orally administered with the administration volume of 10 uL/g, and the vehicle group was administered with the same amount of vehicle, the administration was carried out once per day and lasted for three weeks. During the whole experiment, the tumor sizes of the mice were measured twice each week, and whether or not the presence of toxic reactions was observed. The efficacy was evaluated in terms of relative tumor growth inhibition (TGI).

Tumor Volume (TV) was calculated by: TV=½×a×b×b, where a and b represented the length and the width of tumor, respectively.

Relative tumor growth inhibition (TGI): tumor growth inhibition (TGI) is one of the indicators of tumor response to treatment based on the following equation: TGI (%)=(1−(Tᵢ−T₀)/(Vᵢ−V₀))×100%,

Tᵢ: the average tumor volume of the experimental group corresponding to the analysis day number; T₀: the average tumor volume of the experimental group corresponding to the day on which the animals were grouped; Vᵢ: the average tumor volume of the vehicle group corresponding to the analysis day number; and V₀: the average tumor volume of the vehicle group corresponding to the day on which the animals were grouped.

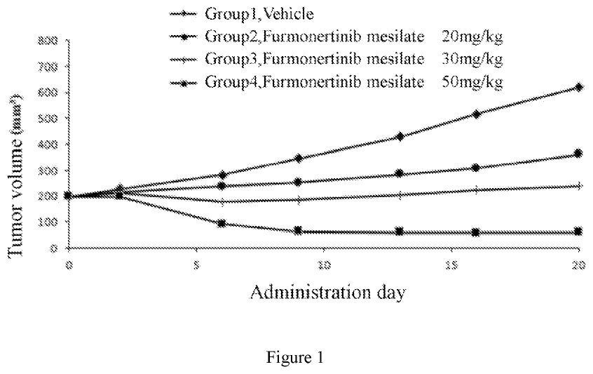

The curves for the tumor volume changes of three experimental groups and one vehicle group were shown in FIG. 1.

The result showed that furmonertinib mesilate showed good anti-tumor effect in HuPrime lung cancer LU0387 subcutaneous xenograft BALB/c female nude mouse animal models.

### 3. Clinical Trial

### a. Protocol

| | |
|---|---|
| Test drug | Furmonertinib mesilate: 40 mg/tablet |
| Test phase | Ib Phase |
| Study | Primary study purposes: |
| purposes | Exploring the therapeutic efficacy of different doses of |
| | furmonertinib in treating locally advanced or metastatic |
| | NSCLC patients with positive EGFR 20 exon insertion |
| | mutation (ORR, Objective response rate) |
| | Secondary study purposes: |
| | Evaluating the therapeutic efficacy of furmonertinib in |
| | locally advanced or metastatic NSCLC patients with positive |
| | EGFR 20 exon insertion mutation |
| | Evaluating the safety and tolerance of different doses of |
| | furmonertinib for treating locally advanced or metastatic |
| | NSCLC patients with positive EGFR 20 exon insertion |
| | mutation. |
| | To explore the correlation between the pharmacokinetics |
| | (PK) and the selected endpoints (possibly including efficacy, |
| | safety and/or quality of life score); to characterize the PK of |
| | furmonertinib mesilate and its metabolite, including the |
| | plasma concentration of furmonertinib mesilate and its |
| | metabolite AST5902; the metabolite to furmonertinib |
| | mesilate ratio of each PK parameter. The effects of internal |
| | factors (gender, age, body weight, hepatic and renal |
| | function, etc.) on PK data will be analyzed using population |
| | PK analysis in this study, which may possibly form a pooled |
| | analysis with the data from other studies |
| | Exploratory study purposes: |
| | To compare EGFR mutation status or possible changes in |
| | drug-resistant gene at baseline, on C5D1 and upon disease |
| | progression using ctDNAs from peripheral blood |
| Study | Primary study endpoints |
| endpoints | ORR |
| | Secondary study endpoints |
| | DCR, DOR, DepOR, PFS, OS |
| | CNS ORR |
| | Safety, including adverse events and severity, |
| | frequency of occurrence, etc |
| Study | Locally advanced or metastatic NSCLC patients with |
| objects | positive EGFR 20 exon insertion mutation, confirmed by |
| | examination, who either has received no prior systematic |
| | anti-tumor therapy or has progressive disease after receiving |
| | prior systematic anti-tumor therapy. |
| Cohorts | Furmonertinib mesilate, administered daily, one treatment |
| | cycle period every 3 weeks. 3 treatment groups in total. |
| | Test group 1: previously-treated subjects received |
| | furmonertinib at 160 mg/day, orally administered under |
| | fasted state, once per day, until disease progression, |
| | death or intolerance. |
| | Test group 2: previously-treated subjects received |
| | furmonertinib at 240 mg/day, orally administered under |
| | fasted state, once per day, until disease progression, |
| | death or intolerance. |
| | Test group 3: treatment-naive subjects received |
| | furmonertinib at 240 mg/day, orally administered under |
| | fasted state, once per day, until disease progression, |
| | death or intolerance. |
| | Note: It was allowable to adjust the dose if |
| | treatment-related adverse events occurred |
| Inclusion | Patients must meet all of the following inclusion criteria to |
| criteria | be enrolled in the study: |
| | 1. Greater than or equal to 18 years old, male or female; |
| | 2. Having histologically or cytopathologically confirmed |
| | primary non-small cell lung cancer (NSCLC) with |
| | predominant non-squamous cell histology; |
| | 3. Having documented positive EGFR exon 20 insertion |
| | mutation by laboratory test, after the disease imageological |
| | progression in the last systematic anti-tumor therapy, for the |
| | previously-treated patients, and before joining the group, for |
| | the treatment-naive patients; |
| | 4. The subject met either of the following two conditions |
| | (lung cancer staging criteria according to AJCC version 8): |
| | 1) Previously-treated patients: locally advanced (judged by |
| | the investigator as inappropriate for surgery or radiation |
| | therapy) or metastatic NSCLC patients, confirmed to have |
| | imageological or pathological disease progression during or |
| | after the last systematic anti-tumor therapy prior to the start |
| | of treatment |
| | 2) Treatment-naive patients: locally advanced (judged by the |
| | investigator as inappropriate for surgery or radiation |
| | therapy) or metastatic NSCLC patients, having received no |
| | prior systematic anti-tumor therapy prior to the start of |
| | treatment. |
| | 5. Having at least one measurable lesion (according to |
| | RECIST1.1). |
| | 6. Laboratory test showing the subjects have adequate organ |
| | function; |
| | 7. Having an ECOG score of 0-1 upon screening and no |
| | obvious disease deterioration within 2 weeks before |
| | screening; |
| | 8. Life expectancy >12 weeks after the start of treatment; |
| | 9. Female subjects of childbearing potential must have a |
| | negative serum pregnancy test within 7 days prior to the start |
| | of treatment; female subjects of non-childbearing potential |
| | may not undergo pregnancy test or contraception. |
| | Non-childbearing potential is defined as: at age of 50 years |
| | and older, no use of hormone therapy and amenorrhea for at |
| | least 12 months; or having received sterilization operation. |
| | Female subjects at childbearing age and male subjects agree |
| | to take effective contraceptive measures during the study |
| | and within 6 months after drug discontinuation; |
| | 10. Being able to understand and voluntarily participate in |
| | the study, and sign the informed consent form. |
| Exclusion | Patients who meet any one of the following criteria cannot |
| Criteria | be enrolled in the study. |
| | 1. NSCLC with predominant squamous cell histology, small |
| | cell lung cancer or neuroendocrine carcinoma indicated by |
| | histology or cytology test; |
| | 2. Expected to receive other anti-tumor therapy other than |
| | furmonertinib during the treatment; |
| | 3. Treatment-naive patients: having previously received |
| | systematic anti-tumor therapy targeting EGFR pathway |
| | (marketed drugs or drugs under development, EGFR TKI or |
| | antibody, etc.) |
| | Previously-treated patients: having previously received |
| | systematic anti-tumor therapy with third-generation EGFR |
| | TKI (marketed drugs or drugs under development); |
| | 4. Having received the following therapies: |
| | 1) Having been irradiated for >30% bone marrow or a large |
| | area within 4 weeks prior to the start of treatment; |
| | 2) Having received major surgery within 4 weeks prior to the |
| | start of treatment or plan to receive major surgery during the |
| | study with exception of the surgical procedures to establish |
| | vascular access, biopsy through mediastinoscopy or |
| | thoracoscopy; |
| | 3) Use of a potent CYP3A4 inhibitor within 7 days prior to |
| | the start of treatment or a potent CYP3A4 inducer within 21 |
| | days prior to start of treatment; a traditional Chinese |
| | medicine or traditional Chinese medicine preparation |
| | indicated for tumor, or a traditional Chinese medicine or |
| | traditional Chinese medicine preparation with adjuvant |
| | anti-tumor effects has been used within 2 weeks prior to the |
| | start of treatment or is expected to be used during the |
| | treatment; |
| | 4) Having participated in the clinical trial and received |
| | furmonertinib within 4 weeks or at least 5 half-lives prior to |
| | the start of treatment; |
| | 5) Having received other anti-tumor drugs within 14 days |
| | prior to the start of treatment; |
| | 5. Concurrent spinal cord compression or symptomatic brain |
| | metastasis. Subjects with stable brain metastasis will be |
| | eligible. Stable brain metastasis is defined as the patients |
| | who have completed regular treatment for brain metastasis, |
| | are clinically stable or asymptomatic for at least 2 weeks and |
| | do not need steroid therapy. If the investigator considers |
| | there is no indication of immediate radical treatment, |
| | patients with asymptomatic brain metastasis will be eligible. |
| | 6. The toxicity caused by previous anti-tumor therapy has |
| | not recovered to ≤CTCAE grade 1 (CTCAE 5.0) (except |
| | alopecia, sequelae of previous platinum-related |
| | neurotoxicity) or the level specified in the |
| | inclusion/exclusion criteria; |
| | 7. Unstable pleural effusion or peritoneal effusion with |
| | obvious symptoms; those with stable clinical symptoms for |
| | at least 28 days after drainage of pleural effusion or ascites |
| | will be eligible; |
| | 8. Having a history of other malignant tumor, or other |
| | concurrent malignant tumors (except those that have |
| | undergone radical operation and have no recurrence within 5 |
| | years post operation, e.g., cervical carcinoma in situ, basal |
| | cell carcinoma of skin and papillary thyroid carcinoma); |
| | 9. Previous interstitial lung disease (ILD), drug-induced |
| | interstitial lung disease, radiation pneumonitis requiring |
| | steroid therapy; or having the clinical manifestations of |
| | suspected interstitial lung disease; |
| | 10. Having severe or uncontrolled systemic disease requiring |
| | treatment that is considered by the investigator as ineligible |
| | for the study, including hypertension, diabetes, chronic heart |
| | failure (NYHA Functional Classification III-IV), unstable |
| | angina pectoris, myocardial infarction within 1 year, and |
| | active hemorrhagic disease, etc.; |
| | 11. Left ventricular ejection fraction (LVEF) <50% on |
| | echocardiography; |
| | 12. Clinically significant prolonged QT interval or other |
| | arrhythmia or clinical status considered by the investigator |
| | that may increase the risk of prolonged QT interval; for |
| | example, QTc > 470 ms on ECG at resting state, complete |
| | left bundle branch block, third-degree atrioventricular block, |
| | congenital long QT syndrome, serious hypokalemia, or |
| | current use of drugs that may lead to prolonged QT interval; |
| | 13. Serious gastrointestinal dysfunction, or disease that may |
| | affect the intake, transport or absorption of furmonertinib; |
| | 14. Known hepatitis B virus (positive HBsAg), hepatitis C |
| | virus (positive HCV Ab) or human immunodeficiency virus |
| | (positive HIV antibody) infection; |
| | 15. Infectious disease requiring intravenous medication; |
| | 16. Known history of mental disease or drug abuse, and |
| | currently having an attack or still taking drugs; |
| | 17. Known or suspected allergy to furmonertinib or other |
| | components of its preparation; |
| | 18. Female subjects or female partners of male subjects who |
| | are pregnant or lactating, or plan to be pregnant during the |
| | study; |
| | 19. Poor compliance, and inability to comply with the study |
| | procedures, restrictions or requirements; |
| | 20. Other conditions that are considered by the investigator |
| | as unsuitable to participate in this study. |
| Sample | 30 subjects were enrolled into 3 cohorts (160 mg/day for |
| size | previously-treated subjects, 240 mg/day for |
| | previously-treated subjects and 240 mg/day for |
| | treatment-naive subjects), 10 patients in each cohort. The |
| | previously-treated subjects randomly came into the 160 |
| | mg/day or 240 mg/day dose group. |
| Efficiency | Tumor image evaluation |
| evaluation | Image evaluation was performed according to the evaluation |
| | criteria for solid tumor therapeutic efficacy (RECIST 1.1). |
| | Tumor influence inspection includes computed tomography |
| | (CT) and/or magnetic resonance imaging (MRI). |

### b. Results

| | | | Previously- | Previously- |
|---|---|---|---|---|
| | | Previously- | treated | treated + |
| | Naive | treated | (160 mg + | Naive |
| | (240 mg) | (240 mg) | 240 mg) | (240 mg) |
| ORR | 70%(7/10), | 28.6%(2/7) | 21.4%(3/14) | 52.9%(9/17) |
| DCR | 100%(10/10), | 85.7% | 78.6% | 94.1% |
| | | (6/7) | (11/14) | (16/17) |

Conclusion: Furmonertinib mesilate has a good anti-tumor effect on non-small cell lung cancer (NSCLC) with EGFR exon 20ins mutation at the dose of 160 mg QD., and has an excellent anti-tumor effect on treatment-naive or previously-treated NSCLC with EGFR exon 20ins mutation at the dose of 240 mg QD.

### c. Safety Profile

Long term administration furmonertinib mesilate at the dose of 160 mg-240 mg was well tolerated, and the observed safety events mainly comprised adverse reactions in the gastrointestinal tract and on the skin and laboratory examination abnormity related to liver and kidney; no other specific Adverse Event (AE) categories were observed; AE severity was slight and was mostly CTCAE grade 1-2. Clinical trial showed that the administration at the dose of 160 mg-240 mg for a long time had relatively good safety.

### INDUSTRIAL APPLICABILITY

The present disclosure provides a pharmaceutical composition containing a therapeutically effective amount of furmonertinib or a pharmaceutically acceptable salt thereof and optionally a pharmaceutically acceptable carrier, and use of said pharmaceutical composition in manufacture of a medicament for treating and/or preventing a disease mediated by EGFR exon 20 insertion mutation. The present disclosure also provides a method of treating and/or preventing a disease mediated by EGFR exon 20 insertion mutation, wherein a therapeutically effective amount of furmonertinib or a pharmaceutically acceptable salt thereof is administered to a patient. The pharmaceutical composition of the present disclosure shows an excellent therapeutic effect on disease mediated by EGFR exon 20 insertion mutation (for example, non-small cell lung cancer (NSCLC)) with little side effects and excellent safety.

1-32. (canceled)

33. A method of treating and/or preventing a disease mediated by EGFR exon 20 insertion mutation, comprising administering to a patient in need thereof a therapeutically effective amount of furmonertinib or a pharmaceutically acceptable salt thereof.

34. (canceled)

35. The method of claim 33, wherein the disease is cancer, such as locally advanced or metastatic non-small cell lung cancer (NSCLC).

36. The method of claim 33, wherein the patient is with confirmed positive EGFR exon 20 insertion mutation.

37. (canceled)

38. The method of claim 36, wherein the patient has received no prior systematic anti-tumor therapy.

39. The method of claim 36, wherein the patient has progressive disease after receiving prior systematic anti-tumor therapy.

40. The method of claim 33, wherein the furmonertinib or the pharmaceutically acceptable salt thereof is administered_at a dose of 80 mg-400 mg.

41. (canceled)

42. The method of claim 33, comprising administering furmonertinib or a pharmaceutically acceptable salt thereof at a dose of 80 mg.

43. The method of claim 33, comprising administering furmonertinib or a pharmaceutically acceptable salt thereof at a dose of 160 mg.

44. The method of claim 33, comprising administering furmonertinib or a pharmaceutically acceptable salt thereof at a dose of 240 mg.

45. The method of claim 33, wherein the furmonertinib or a pharmaceutically acceptable salt thereof is administered qd, bid or tid.

46. (canceled)

47. The method of claim 33, wherein the furmonertinib or a pharmaceutically acceptable salt thereof is administered to the patient under fasted state.

48. (canceled)

49. The method of claim 33, wherein the furmonertinib or a pharmaceutically acceptable salt thereof is administered to the patient orally.

50. The method of claim 33, comprising administering furmonertinib in the form of a mesilate salt.

51-59. (canceled)

60. The method of claim 33, wherein the furmonertinib or a pharmaceutically acceptable salt thereof is administered to the patient before or after surgical resection of tumor.

61-62. (canceled)

63. The method of claim 33, wherein the EGFR exon 20 insertion mutation is at least one selected from EGFR D770_N771insX mutation, EGFR V769_D770insX mutation, EGFR H773_V774insX mutation and EGFR P772_H773insX mutation, such as at least one selected from EGFR D770_N771insSVD, EGFR V769_D770insASV, EGFR H773_V774insNPH and EGFR D770_N771insNPG.

64-65. (canceled)

66. The method of claim 33, wherein the patient has histologically or cytopathologically confirmed primary non-small cell lung cancer (NSCLC) with predominant non-squamous cell histology prior to the start of treatment with furmonertinib or a pharmaceutically acceptable salt thereof.

67-70. (canceled)

71. The method of claim 33, wherein the patient has at least one measurable lesion prior to the start of treatment with furmonertinib or a pharmaceutically acceptable salt thereof.

72. The method of claim 33, wherein the patient has adequate organ function as shown by laboratory test prior to the start of treatment with furmonertinib or a pharmaceutically acceptable salt thereof.

73. The method of claim 33, wherein the patient has an ECOG PS (Eastern Cooperative Oncology Group performance status) score of 0-1 prior to the start of treatment with furmonertinib or a pharmaceutically acceptable salt thereof.

74-77. (canceled)

78. A method of for providing tumor shrinkage in target lesions as evaluated by tumor radiological examination, such as computed tomography (CT) and/or magnetic resonance imaging (MRI), the method comprising administering to a patient in need thereof a therapeutically effective amount of furmonertinib or a pharmaceutically acceptable salt thereof.
